(19) United States
(12) Patent Application Publication
(10) Pub. No.: US 2025/0144380 A1
(43) Pub. Date: May 8, 2025

(54) APPARATUS AND METHOD FOR AUTOMATICALLY MANUFACTURING BALLOON CATHETER TUBES

(71) Applicant: Je-Kwon Lee, Seoul (KR)

(72) Inventor: Je-Kwon Lee, Seoul (KR)

(21) Appl. No.: 18/923,811

(22) Filed: Oct. 23, 2024

(30) Foreign Application Priority Data
Nov. 2, 2023 (KR) 10-2023-0149937

(51) Int. Cl. A61M 25/10

(52) U.S. Cl. CPC A61M 25/1036

(57) ABSTRACT

The present invention provides apparatuses and methods for automatically manufacturing balloon catheter tubes, and it is an object of the present invention is to provide apparatuses and methods for automatically manufacturing balloon catheter tubes, which prevent the catheter tube from being bent when inflating the balloon by using a variable extrusion die, mark individual unit sections of a tube being continuously produced by cutting or removing a portion of the tube spaced apart from a section where a rubber adhesion inhibitor is applied by a predetermined length, and efficiently discharge inflation air, thereby preventing accumulation of length measurement errors that may occur in a continuous process and enabling continuous production of the tube.

### CROSS REFERENCE TO RELATED APPLICATIONS

This application claims the benefit of Korean Patent Application No. 10-2023-0149937 filed on Nov. 2, 2023, the entirety of which is incorporated by reference herein.

### BACKGROUND OF THE INVENTION

### 1. Field of the Invention

The present invention relates to apparatuses and methods for automatically manufacturing balloon catheter tubes, and more specifically, to apparatuses and methods for automatically manufacturing balloon catheter tubes, which prevent the catheter tube from being bent when inflating the balloon by using a variable extrusion die, mark individual unit sections of a tube being continuously produced by cutting or removing a portion of the tube spaced apart from a section where a rubber adhesion inhibitor is applied by a predetermined length, and efficiently discharge inflation air, thereby preventing accumulation of length measurement errors that may occur in a continuous process and enabling continuous production of the tube.

### 2. Description of the Related Art

As well known in the art, a balloon catheter is a medical device which is used to insert a tube with a tubular body into a human organ, inflate a balloon using air or liquid injected through an inflation lumen, and fix it inside the human body, and then inject drugs or remove various liquid excretions through a drainage lumen or drug injection lumen additionally formed in the tube.

A typical balloon catheter 2 includes an extruded tube as a main body, as shown in FIG. 1. The extruded tube has a double-tube (2-way) structure in which an inflation lumen 4 for inflation or deflation of the balloon and a drainage lumen 6 for injection of drugs or removal of liquid excretions are extruded as a single tube.

The extruded tube is manufactured by extruding a soft synthetic resin through an extruder. Then, a predetermined portion of the inflation lumen of the extruded tube is perforated, and a separately manufactured balloon is assembled around the perforated portion, followed by bonding and coating to complete the balloon. When forming a separate hole in the drainage lumen for injection of drugs or removal of liquid excretions, major portions of the balloon catheter tube is completed.

Since the above-described method for manufacturing catheter tubes requires high manufacturing costs due to the complicated process, a technique that improves the manufacturing process to eliminate the assembly, bonding, and coating processes of the balloon has already been filed as patents. Representative examples include Korean Patent Registration Nos. 10-0333264, 10-0434720, 10-0689238, 10-1922800, 10-2168072, 10-2056983 and the like.

When dividing the above patents according to characteristics of the tube structure, it is possible to classify them into Group 1 (Patent Registration Nos. 10-0333264, 10-0434720 and 10-0689238), and Group 2 (Patent Registration Nos. 10-1922800, 10-2168072 and 10-2056983). The patents of Group 1 have a structure in which the inflation lumen of the catheter tube exists inside the tube, such that a separate operation of perforating the tube is required to connect the inflation lumen and the balloon duct, and the patents of Group 2 have the characteristic of not requiring a separate perforation operation, because the inflation lumen is exposed to an outside of the inner tube.

In the patents of Group 1, the catheter tube is extruded for the perforation operation, and then cut to separate it into individual units before performing a subsequent operation. Therefore, it is a production method not capable of producing tubes through a continuous process, such that the production costs should be higher than those of Group 2. On the other hand, in the patents of Group 2, the perforation operation is omitted, and continuous operation is possible, but due to the structure of the inflation lumen in which the inner tube is exposed to the outside, a phenomenon where the catheter tube is bent when inflating the catheter balloon occurs. This phenomenon occurs when the relatively hard inner tube fails to symmetrically offset a longitudinal tensile force generated while the soft catheter balloon is inflated. In other words, in terms of the cross-sectional structure, in the patents of Group 2, due to the structure of the inflation lumen in which the inner tube is exposed to the outside, an inner tube surface on the inflation lumen side does not have a structure that can withstand the longitudinal tensile force. Therefore, the inner tube surface is longer during extending than the side opposite to the inflation lumen where the structure exists. As a result, the catheter tube is bent in a direction opposite to the inflation lumen while the balloon is inflated. This phenomenon tends to be more severe as an outer diameter of the catheter becomes smaller.

The catheter bent as described above may cause unnecessary stimulation inside the patient's organs when used. Considering that small-sized catheters are generally used in children, infants and young patients, the pain of young patients with little patience may be aggravated, thereby resulting in a longer treatment period for the young patients.

Apart from the above-described bending phenomenon problem, the patents of Group 2 have additional problems related to the production process. Generally, balloon catheters use silicone rubber as a raw material, and the silicone rubber tube, which is a thermosetting material, is extruded through an extrusion process and then heated. In this process, the silicone rubber raw material should undergo a crosslinking reaction. In this situation, air existing inside the inflation lumen exposed to the outside of the inner tube in the patents of Group 2 inevitably inflates during the heating process. If the inflating air is not smoothly discharged to front and rear portions of the tube, the coating material before or during the crosslinking reaction deforms and inflates, thereby making it impossible to produce a tube having the desired shape. The discharge of the inflating air to the rear portion of the tube is continuously and smoothly performed, whereas the discharge of the inflating air to the front portion of the tube becomes difficult as the length of the produced tube is increased due to the increased resistance of line. As a result, good products are produced in the early stages of production, but as the length of the produced tube is increased, it is difficult to discharge the inflating air, such that the coating material for the soft thin balloon is inflated by the inflated air on the inflation lumen side. Therefore, the tube will be produced while the coating material for the balloon is lifted along the inflation lumen.

These problems can be solved by continuously cutting the tube into individual unit tubes at the last stage of the continuous production process, thereby allowing the inflation air to be smoothly discharged to the front portion of the tube. However, the above patents do not describe a method or specific technique for cutting the tube in an automatic production process. In particular, when the tube is cut using a general length measurement method, it is impossible to continuously cut the tube to the desired length due to the accumulated measurement errors of the length measuring device. In addition, since the catheter tube is made of elastic rubber material, even if the length is detected by the most precise measuring device, the measurements become meaningless due to the elasticity of the tube itself. Therefore, minute measurement errors are bound to occur, and in the continuous production method where these errors are accumulated, there is a risk of cutting a portion of the tube that should not be cut.

As described above, these conventional techniques have complicated manufacturing processes and cannot be subjected to continuous production process, which cause an increase in the manufacturing costs. In addition, even in the improved process with high manufacturing efficiency, many problems, such as the phenomenon where the catheter tube is bent when inflating the balloon, occur. Therefore, improvement in these situations is urgently required.

### SUMMARY OF THE INVENTION

In consideration of the above-mentioned circumstances, it is an object of the present invention to provide apparatuses and methods for automatically manufacturing balloon catheter tubes, which prevent the catheter tube from being bent when inflating the balloon by using a variable extrusion die, mark individual unit sections of a tube being continuously produced by cutting or removing a portion of the tube spaced apart from a section where a rubber adhesion inhibitor is applied by a predetermined length, and efficiently discharge inflation air, thereby preventing accumulation of length measurement errors that may occur in a continuous process and enabling continuous production of the tube.

To achieve the above object, according to an aspect of the present invention, there is provided a method for automatically manufacturing balloon catheter tubes, including: a) an inner tube production step of forming an inflation lumen by a predetermined section in a longitudinal direction of an outer circumference of an inner tube by installing a variable extrusion die which is connected to an inner tube extruder and repeatedly performs to define sections including portions where an inflation lumen is formed and not formed; b) a step of repeatedly applying a rubber adhesion inhibitor by recognizing a boundary between the portions where the inflation lumen is formed and not formed of the inner tube; and c) a step of performing coating and extrusion of a balloon material on an outside of the inner tube to manufacture longitudinal balloon catheter tubes in which balloons are repeatedly formed.

Preferably, the application of the rubber adhesion inhibitor in step b) is performed by a non-contact application using a spray and a screen, or a stamping-type contact application for directly transferring a stamp to which the rubber adhesion inhibitor is adhered to a surface of the inner tube.

According to another aspect of the present invention, there is provided a method for automatically manufacturing balloon catheter tubes, including: a) a step of cutting or removing an inner tube partial portion D-D′ of a portion spaced apart from a position where a rubber adhesion inhibitor is applied to a surface of the inner tube being continuously produced to mark a section of the catheter tube; b) a step of continuously performing coating and extrusion of a balloon material on an outside of the inner tube, from which the portion is cut or removed; and c) a step of detecting the cut or removed portion of the inner tube of the catheter tube on which the balloon material is coated and extruded to finally cut the catheter tube by a predetermined section.

Preferably, the cutting or removing a portion of the inner tube in step a) is performed simultaneously with the application of the rubber adhesion inhibitor.

Preferably, the cutting or removing a portion of the inner tube in step a) is performed by cutting or removing the portion of the inner tube in a curved shape such as a fan or semicircular shape.

Preferably, the cutting a portion of the inner tube in step a) is performed by cutting or removing the portion of the inner tube so as to mutually penetrate an inflation lumen and a drainage lumen.

In addition, according to another aspect of the present invention, there is provided an apparatus for automatically manufacturing balloon catheter tubes, including: an inner tube extruder having a variable extrusion die installed therein to manufacture an inner tube in which portions where an inflation lumen is formed and not formed are repetitively defined; a rubber adhesion inhibitor application device provided at a rear end of the inner tube extruder, and configured to repeatedly applying a rubber adhesion inhibitor to a boundary between the portions where the inflation lumen is formed and not formed of the inner tube; and a coating extruder configured to extrude a coating layer on an outer circumference of the inner tube that has passed through the rubber adhesion inhibitor application device.

Preferably, the variable extrusion die of the inner tube extruder is connected to a pneumatic cylinder, a hydraulic cylinder or a motor, and is configured so that the position of the variable extrusion die is controlled in conjunction with an extrusion tube length measuring device or a timer.

Preferably, the rubber adhesion inhibitor application device includes any one of a non-contact application device including a plurality of rubber adhesion inhibitor sprays installed at a position spaced apart from the inner tube by a predetermined distance and a screen close to the inner tube, or a stamping-type contact application device equipped with a stamp configured to hold a rubber adhesion inhibitor and repeatedly transfer it to a predetermined portion of an outer circumference of the inner tube.

Further, according to another aspect of the present invention, there is provided an apparatus for automatically manufacturing balloon catheter tubes, including: a partial cutter configured to repeatedly cut and remove a portion of an inner tube being produced; a coating extruder positioned at a rear end of the partial cutter and configured to extrude a balloon coating layer on an outer circumference of the partially cut inner tube; and a final cutter positioned at a rear end of the coating extruder or in the next process, and configured to cut a catheter tube by detecting a position where the inner tube is partially cut by contacting the outer circumference of the catheter tube being produced or through a vision sensor.

Preferably, the partial cutter is operated to cut the portion of the inner tube by detecting a position of the portion by receiving an operation signal from a timer, a tube extrusion length measurement device, or another device such as a rubber adhesion inhibitor application device.

Preferably, the partial cutter cuts or removes the portion of the inner tube using a circular or curved blade.

Preferably, the partial cutter is controlled to prevent the inner tube from being twisted in the partial cutter through a guide having a protrusion which is inserted into an inflation lumen of the inner tube, or prevent the inner tube from being twisted through a vision sensor.

The apparatus and method for automatically manufacturing balloon catheter tubes according to the present invention define portions where an inflation lumen is formed or not formed using a variable extrusion die, thereby preventing the catheter from being bent when inflating the balloon, and mark a tube section on the inner tube and cut the finished product based on the marked section, thereby preventing the accumulation of errors in length measurement that may occur due to continuous production, and enabling continuous production of good products.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a cross-sectional view illustrating a conventional balloon catheter tube having a double-tube structure of the prior art;

FIG. 2 is a view illustrating the structure of a variable extrusion die included in an apparatus for automatically manufacturing balloon catheter tubes according to an embodiment of the present invention;

FIG. 3 is a cross-sectional view illustrating an inner tube portion A, where an inflation lumen is formed, manufactured by the variable extrusion die shown in FIG. 2;

FIG. 4 is a cross-sectional view illustrating an inner tube portion B, where an inflation lumen is not formed, manufactured by the variable extrusion die shown in FIG. 2;

FIG. 5 is a perspective view illustrating the shape of an inner tube in which inflation lumens are repeatedly formed and not formed through the apparatus for automatically manufacturing balloon catheter tubes according to an embodiment of the present invention;

FIG. 6 is a perspective view illustrating a state where a rubber adhesion inhibitor is applied through the apparatus for automatically manufacturing balloon catheter tubes according to an embodiment of the present invention;

FIG. 7 is a perspective view illustrating a state where the inner tube is partially cut through the apparatus for automatically manufacturing balloon catheter tubes according to an embodiment of the present invention;

FIG. 8 is a view illustrating a working table having an inner tube twisting rotation prevention guide formed in the apparatus for automatically manufacturing balloon catheter tubes according to an embodiment of the present invention;

FIG. 9 is a view illustrating a state where a partially cut inner tube is coated and extruded through the apparatus for automatically manufacturing balloon catheter tubes according to an embodiment of the present invention; and

FIG. 10 is a view illustrating a state where a tube manufactured through the apparatus for automatically manufacturing balloon catheter tubes according to an embodiment of the present invention is cut at a predetermined interval.

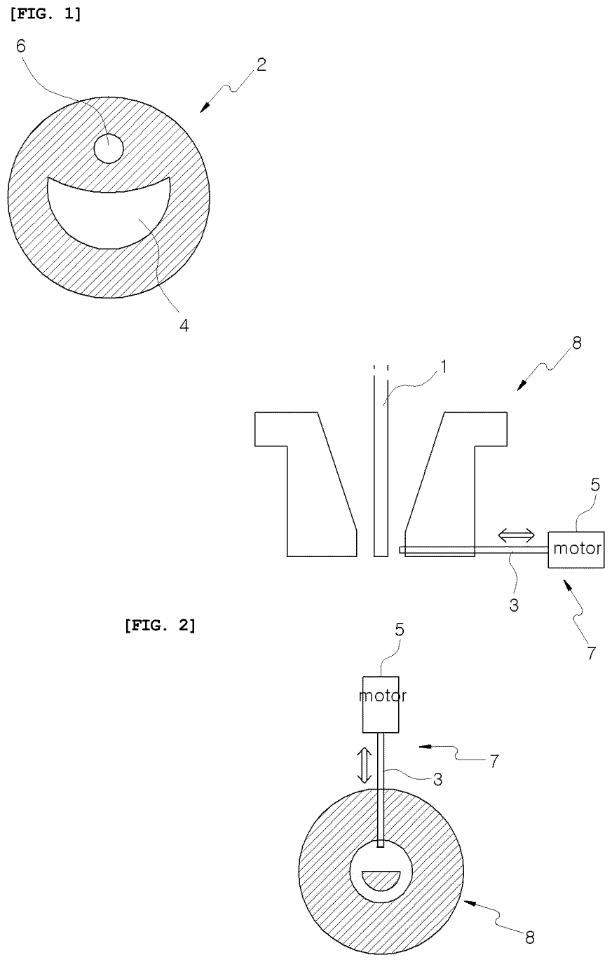

### DETAILED DESCRIPTION OF THE INVENTION

Hereinafter, the present invention will be described in detail with reference to the drawings.

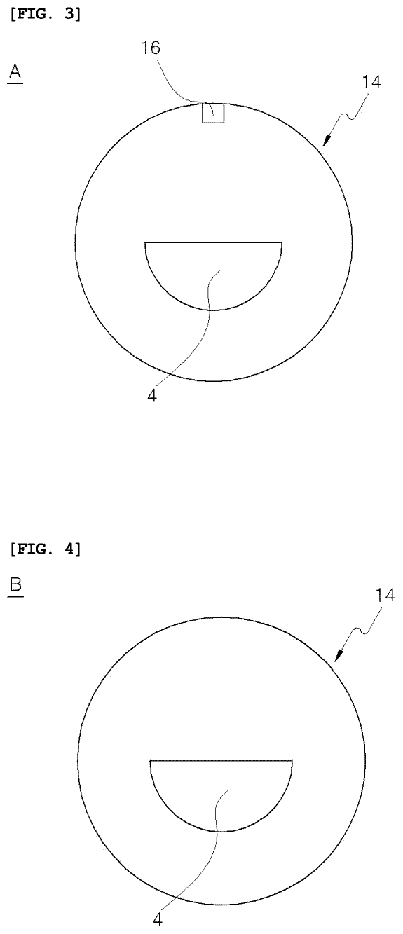

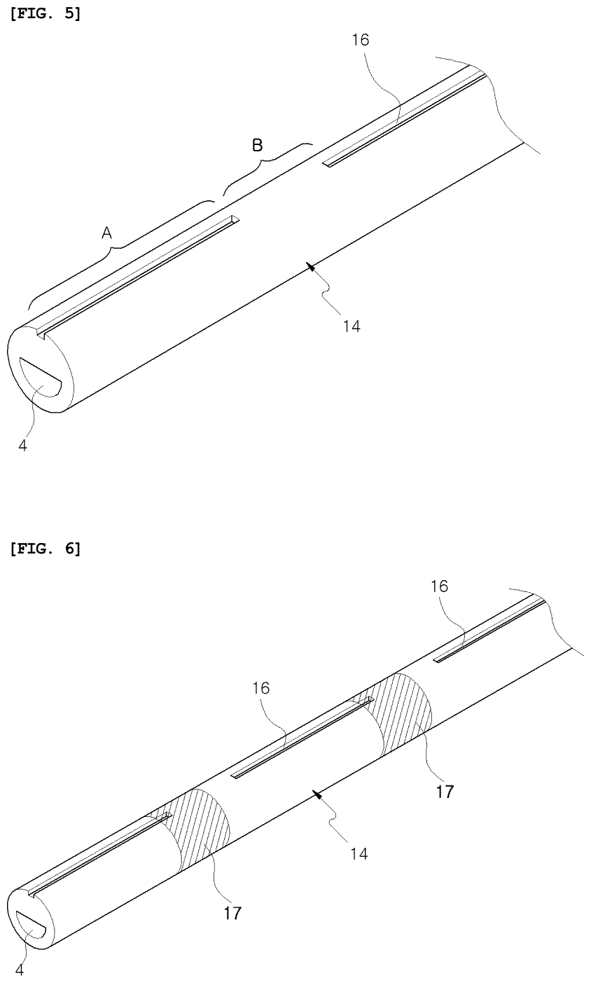

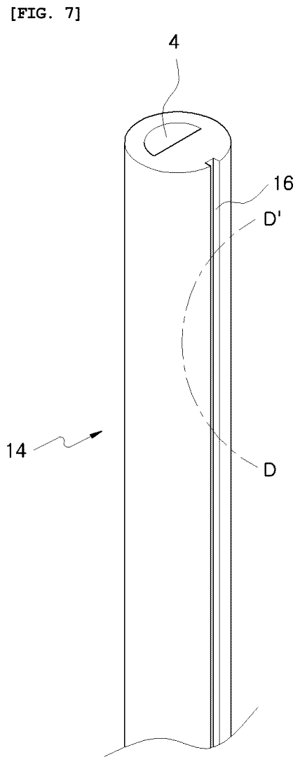

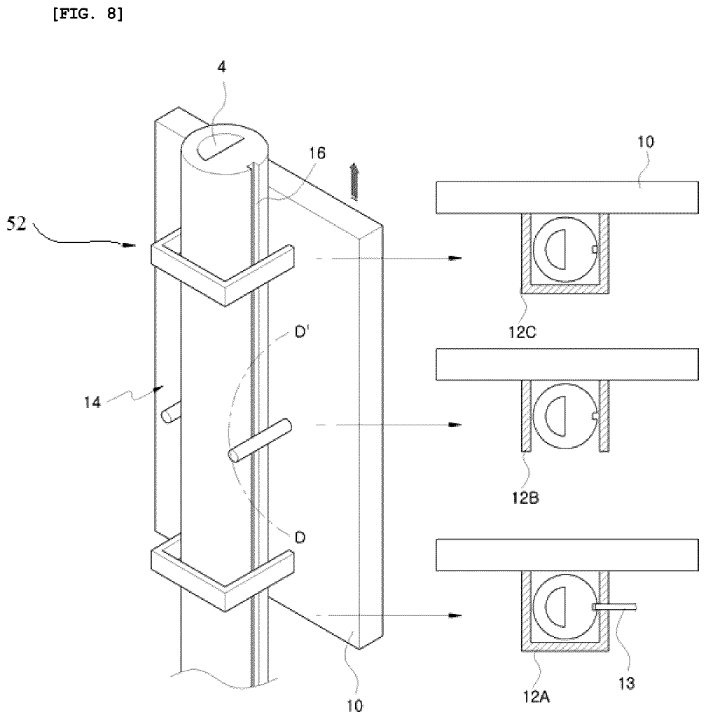

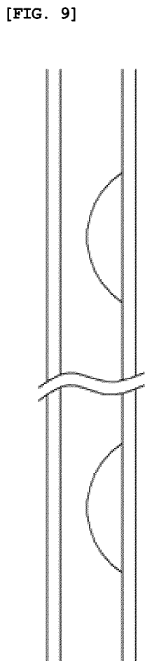

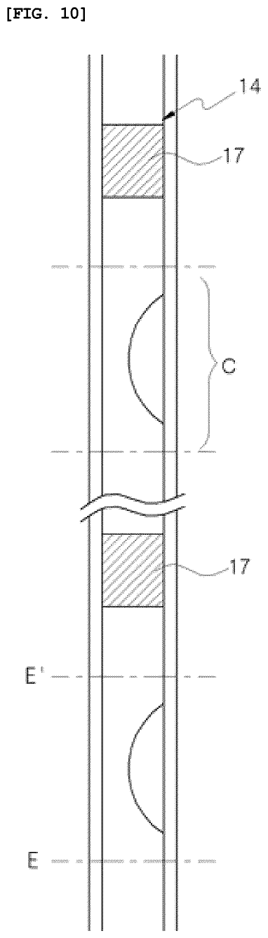

FIG. 2 is a view illustrating the structure of a variable extrusion die included in an apparatus for automatically manufacturing balloon catheter tubes according to an embodiment of the present invention; FIG. 3 is a cross-sectional view illustrating an inner tube portion A, where an inflation lumen is formed, manufactured by the variable extrusion die shown in FIG. 2; FIG. 4 is a cross-sectional view illustrating an inner tube portion B, where an inflation lumen is not formed, manufactured by the variable extrusion die shown in FIG. 2; FIG. 5 is a perspective view illustrating the shape of an inner tube in which inflation lumens are repeatedly formed and not formed through the apparatus for automatically manufacturing balloon catheter tubes according to an embodiment of the present invention; FIG. 6 is a perspective view illustrating a state where a rubber adhesion inhibitor is applied through the apparatus for automatically manufacturing balloon catheter tubes according to an embodiment of the present invention; FIG. 7 is a perspective view illustrating a state where the inner tube is partially cut through the apparatus for automatically manufacturing balloon catheter tubes according to an embodiment of the present invention; FIG. 8 is a view illustrating a working table having an inner tube twisting rotation prevention guide formed in the apparatus for automatically manufacturing balloon catheter tubes according to an embodiment of the present invention; FIG. 9 is a view illustrating a state where a partially cut inner tube is coated and extruded through the apparatus for automatically manufacturing balloon catheter tubes according to an embodiment of the present invention; and FIG. 10 is a view illustrating a state where a tube manufactured through the apparatus for automatically manufacturing balloon catheter tubes according to an embodiment of the present invention is cut at a predetermined interval.

With reference the drawings, the apparatus for automatically manufacturing balloon catheter tubes according to an embodiment of the present invention is an apparatus for automatically manufacturing balloon catheter tubes, which prevent the catheter tube from being bent when inflating the balloon by using a variable extrusion die, mark individual unit sections of a tube being continuously produced by cutting or removing a portion of the tube spaced apart from a section where a rubber adhesion inhibitor is applied by a predetermined length, and efficiently discharge inflation air, thereby preventing accumulation of length measurement errors that may occur in a continuous process and enabling continuous production of the tube.

The apparatus for automatically manufacturing balloon catheter tubes according to an embodiment of the present invention includes a tube manufacturing mold 8 according to a preferred embodiment, which is provided with an outer mold having a circular hollow portion formed therein. A material 1 extruded through the tube manufacturing mold 8 by an inner tube extruder (not shown) passes through inside of the mold, and in this process, an inner tube 14 is manufactured while an inflation lumen 16 is formed by an inflation lumen sculpting unit 7.

The inner tube extruder has a variable extrusion die installed therein to manufacture an inner tube 14 in which portions where an inflation lumen is formed and not formed are repetitively defined.

At this time, the inflation lumen sculpting unit 7 is driven by a motor 5 and may carve the inflation lumen 16 only in the required section by a lumen sculpting rod 3 which moves to and from the motor 5.

When the inner tube 14 is manufactured using the tube manufacturing mold 8 as described above, the inflation lumen 16 is formed to be exposed to an outside of the inner tube 14, and the inflation lumen is finally completed by a balloon material coating extrusion process, such that a separate air injection perforation process is not necessary and continuous production of the tube is possible.

However, according to a preferred embodiment of the present invention, it is necessary to perform a pretreatment process for applying a rubber adhesion inhibitor to a predetermined portion of the surface of the inner tube 14 before the balloon material coating extrusion process to prevent the coating extrusion material from adhering with the inner tube, and thereby allowing the balloon to inflate. To this end, a rubber adhesion inhibitor application device is located between the inner tube extruder and a balloon coating extruder.

This rubber adhesion inhibitor application device is placed in a process that is performed after the inner tube undergoes a crosslinking reaction, such that a spray type device may also be used. Of course, a method of applying the rubber adhesion inhibitor in a stamping type may also be sufficiently used in the present invention.

The rubber adhesion inhibitor application device includes any one of a non-contact application device including a plurality of rubber adhesion inhibitor sprays installed at a position spaced apart from the inner tube 14 by a predetermined distance and a screen close to the inner tube, or a stamping-type contact application device equipped with a stamp configured to hold a rubber adhesion inhibitor and repeatedly transfer it to a predetermined portion of an outer circumference of the inner tube 14.

Next, when the rubber adhesion inhibitor is applied at a predetermined interval, the inner tube 14 to which the rubber adhesion inhibitor is applied in a form as shown in FIG. 6 may be introduced into a heater for drying the rubber adhesion inhibitor. At this point, the inner tube holds sufficient heat for the crosslinking reaction in advance, such that it can be dried immediately after the rubber adhesion inhibitor is applied. However, in order to reduce a defect rate in the process, the heater may be passed through once more in order to reduce the defect rate in the process after application of the rubber adhesion inhibitor.

A cutter configured to perform partial cutting (“partial cutter”) (not shown) is provided at the front or rear end of the rubber adhesion inhibitor application device. As shown in FIG. 7, the partial cutter may cut a portion of the inner tube 14 at a predetermined position by a predetermined length, thereby smoothly discharging the heated air inside the inner tube 14 that has become too long, and may mark a position where the final cutting should be done as a single product of tube.

The inner tube 14, in which drying and curing of the rubber adhesion inhibitor is completed, is introduced into a coating extruder so that the outside thereof is coated. Since the inner tube 14 has been subjected to drying and curing of the rubber adhesion inhibitor in the previous process, it may be directly introduced into the coating extruder to produce a balloon catheter tube without separate processing.

Then, final cutting is performed as shown in FIG. 10. The cut portion is a section E-E′, which is a section between two vertical points in the longitudinal direction including the partial cut portion D-D′.

More specifically, the tube manufacturing mold 8 having the shape and operation structure shown in FIG. 2 is installed in the inner tube extruder for extruding the inner tube 14, and a position of a core pin of a variable extrusion die, which is connected to an inner tube extruder and repeatedly performs to define sections including portions where an inflation lumen is formed and not formed, is controlled by detecting a length of the inner tube being extruded or by using a pneumatic cylinder, hydraulic cylinder or motor operated in conjunction with a timer. Therefore, the inner tube having cross sections shown in FIG. 3 and FIG. 4 repeatedly formed therein may be continuously produced.

In the shape of the inner tube 14, a portion where the inflation lumen 16 is formed becomes an inflation lumen after the coating extrusion, and a balloon is formed with a portion where the inflation lumen 16 is not formed as a starting point. This shape is intended to provide an inner tube structure that can prevent the inner tube 14 from being bent by the longitudinal tensile force when inflating the balloon.

Next, a boundary between the portions where the inflation lumen 16 is formed/not formed is recognized by detecting the inflation lumen 16 portion of the inner tube 14 being continuously produced using a contact-type probe or a vision sensor, and based on the results, as shown in FIG. 6, a rubber adhesion inhibitor application part 17, in which a rubber adhesion inhibitor is applied to a portion which partially includes a part of the inflation lumen, is formed.

The application of the rubber adhesion inhibitor is performed by a non-contact application using a spray and a screen, or a contact application of a stamping method for directly transferring a stamp to which the rubber adhesion inhibitor is adhered to a surface of the inner tube.

The rubber adhesion inhibitor immediately after application is in a liquid state, and may lose its function if it comes into contact with other portions before drying. Therefore, in order to achieve an efficient manufacturing process through rapid drying, as described above, the inner tube is sufficiently heated by the heater before and after application of the rubber adhesion inhibitor so as to dry it immediately after application in the heated state.

Next, when the inner tube that has been subjected to application and drying of the rubber adhesion inhibitor is introduced into the above-described coating extruder for extruding the balloon coating to coat the surface with the balloon material, the inflation lumen 16, which was open to the outside of the inner tube, becomes a tube shape due to the coating material to function as an inflation lumen, and the rubber adhesion inhibitor application part 17 functions as a balloon because the coating material is not adhered to the inner tube. Therefore, each section is cut to complete the balloon catheter tube.

The inner tube 14 of the balloon portion of the balloon catheter tube produced by the above method has a cross-section as shown in FIG. 4 at most locations except for partial end portions included for inflation and deflation of the balloon.

As shown in the cross-section, since the inner tube 14 is produced in filled with the material on the inflation lumen 16 side, it becomes a shape that can symmetrically withstand the tensile force which occurs in the longitudinal direction when the balloon is inflated. Therefore, it is possible to prevent the catheter tube from being bent due to balloon inflation.

Meanwhile, if the balloon catheter tube subjected to balloon coating extrusion is produced in the form of a roll without cutting each section, the tube can be produced without any particular problem at the beginning of production, but as the length of the production tube is gradually increased, the air inside the inflation lumen that inflates due to the curing heat is not smoothly discharged. Eventually, the coating material that has not completed the crosslinking reaction is deformed, and a defective product is produced in which the balloon coating is deformed in a lifted state along the inflation lumen in the longitudinal direction.

In addition, to prevent this problem, if automatic cutting of the balloon catheter tube in each section is performed in conjunction with length detection or a timer, a minute error in the measuring instrument, an error between the measured value and the actual length due to the elasticity of the tube itself, or an error between the timer and the actual extrusion speed will occur.

Although individual errors are minimal and do not cause major problems in the early stage of they occur, if these errors are accumulated due to the continuous production, unintended parts will eventually be cut, thereby resulting in an occurrence of a large amount of defective products.

To prevent this problem, by partially cutting a portion of the inner tube spaced apart from the portion to which the rubber adhesion inhibitor is applied as shown in FIG. 7 using the apparatus for automatically manufacturing balloon catheter tubes according to an embodiment of the present invention, the cut portion D-D′ is removed.

The cutting or removing a portion of the inner tube 14 is performed simultaneously with the application of the rubber adhesion inhibitor.

The cutting and removal of the above portion is performed so as to penetrate the inflation lumen 16 formed on the outside and a drainage lumen 4 formed on the inside as shown in FIG. 7. In addition, to prevent the inner tube from being ruptured by the tensile force of the tube that may occur in the production process, the above portion is cut and removed in a curved shape such as a semicircular or fan shape without sharp portions.

When performing partial cutting so as to penetrate the inflation lumen and the internal drainage lumen, the air which is heated and inflated for the crosslinking reaction of the coating material during the coating extrusion is discharged very smoothly through the drainage lumen 4 as well as the inflation lumen 16, such that the coating process may be performed without causing a deformation of the coating material.

Therefore, when using this method, it is also possible to continuously produce the balloon catheter tube in the form of a roll.

Meanwhile, a curved blade (not shown) of the partial cutter may operate only in a certain direction. Therefore, the inner tube 14 passing through the above portion should be prevented from rotating in the partial cutter so as to continuously perform the cutting and removal operations at the predetermined position, thereby penetrating the inflation lumen 16 and the drainage lumen 4.

In order to prevent rotation of the inner tube, as shown in FIG. 8, when installing a guide having a size which matches the outer diameter of the inner tube 14 and a protrusion which is inserted into the inflation lumen 16 to prevent rotation of the tube on the working table 10, and mounting the partial cutter to operate based on the working table 10, the problem entailed in rotation of the inner tube may be solved.

As illustrated in FIG. 8, a guide unit 52 formed on the working table 10 includes a total of three guides 12A, 12B and 12C vertically installed thereon at an interval. Since the partially cut inner tube 14 also passes through an upper guide 12C in a direction of conveying the tube, if the rotation prevention protrusion 13 is provided therein, catch phenomenon may occur. Therefore, the upper guide 12C is formed as a closed type guide without a protrusion only for maintaining the position of the tube.

A middle guide 12B is installed in the form of having an open front for allowing the blade of the partial cutter (not shown) to move without interference, and tube cutting pieces, which are partially cut and removed from the tube, to be discharged smoothly. A lower guide 12A is formed as a closed type guide having a protrusion 13 for holding the position of the tube and preventing the tube from being twisted and rotated.

When cutting and removing the portion D-D′ of the tube by operating a circular or fan-shaped blade of the partial cutter installed in the above-described type working table 10, it is possible to continuously perform cutting of the portion through which the inflation lumen 16 and the drainage lumen 4 penetrate.

In addition, as another method, the purpose of the present invention may be sufficiently achieved by controlling the rotational position of the inner tube 14 by directly checking the inflation lumen 16 or a contrast line with a vision sensor.

Balloon catheter tubes are commonly manufactured by embedding a contrast line capable of being detected with X-rays in the catheter tube to protect against catheter rupture accidents that may occur in the body of a patient.

By installing a mechanism which detects this portion with a vision camera (not shown) or directly detects the inflation lumen 16, and operates to compensate when the portion deviates from the desired position, it is possible to prevent the tube form being rotated in the partial cutter.

When performing partial cutting in this way and performing balloon coating through the coating extrusion process, a tube is produced in the form as shown in FIG. 10. Thereafter, as shown in FIG. 10, the section E-E′ between both points is finally cut using the partially cut portion as a reference point, then the partially cut section is discarded, and the remaining sections are aligned as good products, such that the balloon catheter tube is finally completed.

In the final cutting process, each section may be detected using a roller equipped with a spring or an air cylinder roller equipped with a regulator on the tube surface that is coated and conveyed in the form shown in FIG. 10.

That is, when the tube is conveyed while applying an external force to the tube surface with a constant force, positions of the roller in the non-partially cut section and the partially cut section are different from each other. When detecting the difference, each section of the tube being conveyed may be determined, and the final cutting is performed as shown in FIG. 10 using the partially cut portion as the reference point.

A cutter configured to perform final cutting (“final cutter”) may be provided together with the entire facility according to an embodiment of the present invention to produce the balloon catheter tubes in individual units. Alternatively, the tubes may be produced in the form of a roll and supplied to the next process in the form of a long continuous tube, as well as the final cutter may be provided separately to perform the final cutting in the next process.

The final cutter is positioned at a rear end of the coating extruder or in the next process, and is configured to cut a catheter tube by detecting a position where the inner tube is partially cut by contacting the outer circumference of the catheter tube being produced or through a vision sensor.

Meanwhile, the apparatus and method for automatically manufacturing balloon catheter tubes according to an embodiment of the present invention are not limited to above-described embodiments, but may be variously modified without departing from the technical gist of the present invention.

### DESCRIPTION OF REFERENCE NUMERALS

4: Drainage lumen
10: Working table
14: Inner tube
16: Inflation lumen
17: Rubber adhesion inhibitor application part

What is claimed is:

1. A method for automatically manufacturing balloon catheter tubes, comprising:
a) an inner tube production step of forming an inflation lumen by a predetermined section in a longitudinal direction of an outer circumference of an inner tube by installing a variable extrusion die which is connected to an inner tube extruder and repeatedly performs to define sections including portions where an inflation lumen is formed and not formed;
b) a step of repeatedly applying a rubber adhesion inhibitor by recognizing a boundary between the portions where the inflation lumen is formed and not formed of the inner tube; and
c) a step of performing coating and extrusion of a balloon material on an outside of the inner tube to manufacture longitudinal balloon catheter tubes in which balloons are repeatedly formed.

2. The method for automatically manufacturing balloon catheter tubes according to claim 1, wherein the application of the rubber adhesion inhibitor in step b) is performed by a non-contact application using a spray and a screen, or a stamping-type contact application for directly transferring a stamp to which the rubber adhesion inhibitor is adhered to a surface of the inner tube.

3. A method for automatically manufacturing balloon catheter tubes, comprising:
a) a step of cutting or removing an inner tube partial portion D-D′ of a portion spaced apart from a position where a rubber adhesion inhibitor is applied to a surface of the inner tube being continuously produced to mark a section of the catheter tube;
b) a step of continuously performing coating and extrusion of a balloon material on an outside of the inner tube, from which the portion is cut or removed; and
c) a step of detecting the cut or removed portion of the inner tube of the catheter tube on which the balloon material is coated and extruded to finally cut the catheter tube by a predetermined section.

4. The method for automatically manufacturing balloon catheter tubes according to claim 3, wherein the cutting or removing a portion of the inner tube in step a) is performed simultaneously with the application of the rubber adhesion inhibitor.

5. The method for automatically manufacturing balloon catheter tubes according to claim 3, wherein the cutting or removing a portion of the inner tube in step a) is performed by cutting or removing the portion of the inner tube in a curved shape such as a fan or semicircular shape.

6. The method for automatically manufacturing balloon catheter tubes according to claim 3, wherein the cutting a portion of the inner tube in step a) is performed by cutting or removing the portion of the inner tube so as to mutually penetrate an inflation lumen and a drainage lumen.

7. An apparatus for automatically manufacturing balloon catheter tubes, comprising:
an inner tube extruder having a variable extrusion die installed therein to manufacture an inner tube in which portions where an inflation lumen is formed and not formed are repetitively defined;
a rubber adhesion inhibitor application device provided at a rear end of the inner tube extruder, and configured to repeatedly applying a rubber adhesion inhibitor to a boundary between the portions where the inflation lumen is formed and not formed of the inner tube; and
a coating extruder configured to extrude a coating layer on an outer circumference of the inner tube that has passed through the rubber adhesion inhibitor application device.

8. The apparatus for automatically manufacturing balloon catheter tubes according to claim 7, wherein the variable extrusion die of the inner tube extruder is connected to a pneumatic cylinder, a hydraulic cylinder or a motor, and is configured so that the position of the variable extrusion die is controlled in conjunction with an extrusion tube length measuring device or a timer.

9. The apparatus for automatically manufacturing balloon catheter tubes according to claim 7, wherein the rubber adhesion inhibitor application device comprises any one of a non-contact application device including a plurality of rubber adhesion inhibitor sprays installed at a position spaced apart from the inner tube by a predetermined distance and a screen close to the inner tube, or a stamping-type contact application device equipped with a stamp configured to hold a rubber adhesion inhibitor and repeatedly transfer it to a predetermined portion of an outer circumference of the inner tube.

10. An apparatus for automatically manufacturing balloon catheter tubes, comprising:
a partial cutter configured to repeatedly cut and remove a portion of an inner tube being produced;
a coating extruder positioned at a rear end of the partial cutter and configured to extrude a balloon coating layer on an outer circumference of the partially cut inner tube; and
a final cutter positioned at a rear end of the coating extruder or in the next process, and configured to cut a catheter tube by detecting a position where the inner tube is partially cut by contacting the outer circumference of the catheter tube being produced or through a vision sensor.

11. The apparatus for automatically manufacturing balloon catheter tubes according to claim 10, wherein the partial cutter is operated to cut the portion of the inner tube by detecting a position of the portion by receiving an operation signal from a timer, a tube extrusion length measurement device, or another device such as a rubber adhesion inhibitor application device.

12. The apparatus for automatically manufacturing balloon catheter tubes according to claim 10, wherein the partial cutter cuts or removes the portion of the inner tube using a circular or curved blade.

13. The apparatus for automatically manufacturing balloon catheter tubes according to claim 10, wherein the partial cutter is controlled to prevent the inner tube from being twisted in the partial cutter through a guide having a protrusion which is inserted into an inflation lumen of the inner tube, or prevent the inner tube from being twisted through a vision sensor.
